(19) United States
(12) Patent Application Publication
(10) Pub. No.: US 2010/0129927 A1
(43) Pub. Date: May 27, 2010

(54) Peptides for Use in Diagnosing the Presence of Ruptured Atherosclerotic Lesions in a Individual

(75) Inventors: Catharina Barbara Josephina Maria Cleutjens, Maastricht (NL); Matthias Joseph Alphons Pieter Daemen, Maastricht (NL)

Correspondence Address: MORGAN, LEWIS & BOCKIUS, LLP (SF), ONE MARKET SPEAR STREET TOWER, SAN FRANCISCO, CA, 94105, US

(73) Assignee: UNIVERSITEIT MAASTRICHT/CARIM, Maastricht (NL)

(21) Appl. No.: 12/299,727

(22) PCT Filed: May 4, 2007
(86) PCT No.: PCT/EP2007/054373

§ 371 Date: Sep. 15, 2009

(30) Foreign Application Priority Data
May 5, 2006 (EP) 06009314.3

(51) Int. Cl. G01N 33/53; C07K 7/08; C07K 14/00

(52) U.S. Cl. 436/501; 530/327; 530/326; 530/324; 530/350

(57) ABSTRACT

The present invention relates to peptides that are able to interact with ruptured atherosclerotic plaque-associated antibodies, which peptides comprise an immunoreactive part of one of the amino acid sequences according to SEQ ID NOS: 1-25. The invention further relates to a diagnostic reagent comprising the peptides, to the use of the method and to a method and test kit for the diagnosis of a sample from an individual for the presence therein of antibodies that are indicative of the presence of ruptured atherosclerotic plaques in the individual.

The present invention relates to peptides that interact with ruptured atherosclerotic plaque-associated antibodies in an individual and to their use in diagnostic reagents and diagnostic assays for determining the presence in an individual of ruptured atherosclerotic lesions.

Atherosclerosis is a chronic inflammatory disease of the arteries and is characterized by the accumulation of lipids, cells, cellular waste products, extracellular matrix and calcification in the inner lining (the intima) of an artery. This buildup forms the so-called atherosclerotic plaque.

Cardiovascular disease, currently the leading cause of death and illness in Western Society, will soon become the pre-eminent health problem worldwide. The vast majority of acute clinical manifestations including Myocardial Infarction (MI) and stroke are not due to progressive luminal narrowing due to the presence of a slowly growing, stable, atherosclerotic plaque, but are caused by the formation of an occluding thrombus on the surface of an unstable plaque.

Morphological criteria to discriminate between stable and unstable plaques are well-described and include the presence of a large lipid core, a thin fibrous cap, erosion of the endothelium and the presence of an inflammatory process inside the fibrous cap or the shoulder region of unstable plaques.

The major limitation of these morphological criteria is that they can only be applied after an interventional procedure. As a result, markers to identify unstable or ruptured plaques non-invasively and a fast, sensitive and specific in vitro test for the identification of patients having ruptured or rupture prone lesions is not available.

Despite the increasing knowledge on the pathogenesis of atherosclerosis and the suggestion that several circulating markers of inflammation, such as C-reactive protein, fibrogen and interleukins, are associated with cardiovascular disease, biomarkers for non-invasive diagnosis of ruptured atherosclerotic lesions and a high prognostic value for the identification of individual patients at high risk of future cardiovascular events are not readily available.

It is therefore the object of the present invention to provide such biomarkers for non-invasive diagnosis of ruptured atherosclerotic lesions.

In the research that led to the invention peptides were identified that bind specifically to antibodies that are present in sera of individuals suffering from ruptured plaques.

For the identification of these peptides, a cDNA expression library was used. To create an expression library of cDNAs predominantly expressed in ruptured atherosclerotic lesions, a SSH library of >3000 cDNAs preferentially expressed in ruptured plaques (as described by Faber er al. (Circ Res. 89:547-554 (2001)) was re-cloned and the cDNAs were expressed as fusion proteins with the minor coat protein pVI of filamentous phage M13. To enrich for peptides that bind specifically to antibodies in pooled sera derived from patients known to suffer from ruptured plaques (identified herein as “ruptured sera”), four successive rounds of Serologic Antigen Selection (SAS) were performed. Subsequently, the individual phage clones were tested in ELISA for interaction with sera from individuals suffering from ruptured atherosclerotic plaques.

While the majority of the phage-displayed peptides showed a positive reaction in both pooled sera from patients with ruptured atherosclerotic lesions and pooled sera from patients with stable plaques and healthy controls, the positive reaction of four clones could distinguish pooled sera from patients with ruptured lesions from the sera from patients with stable plaques and control sera (See FIG. 10).

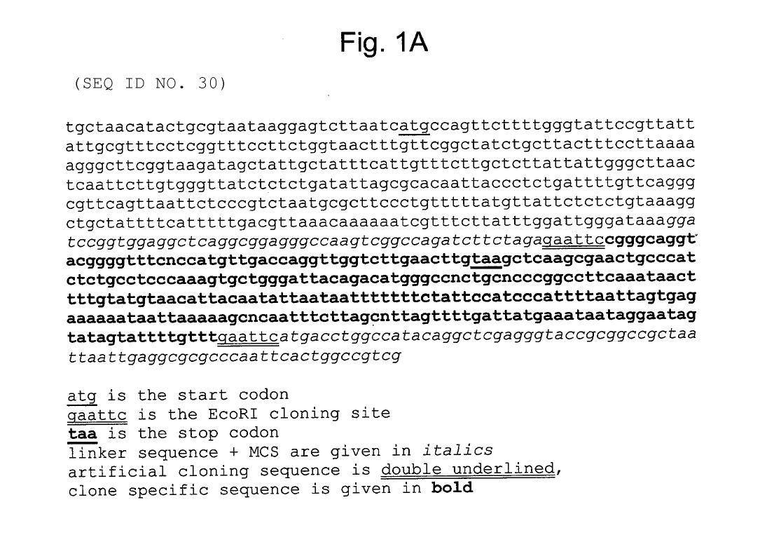

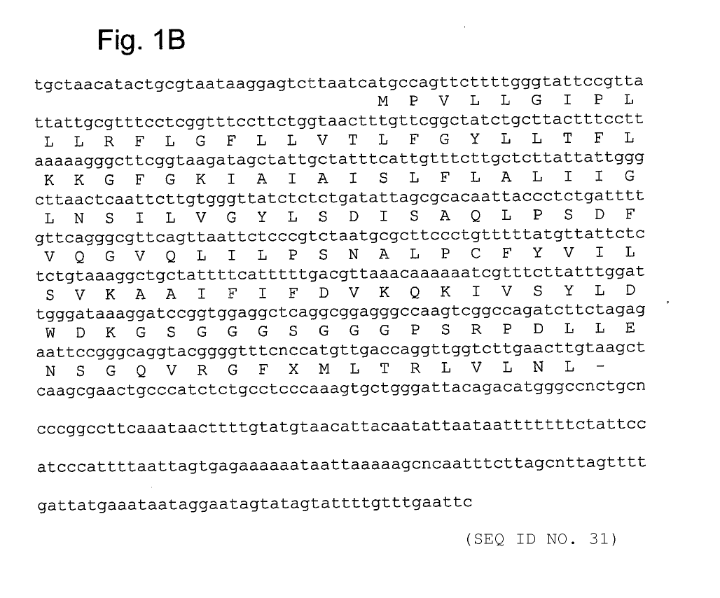

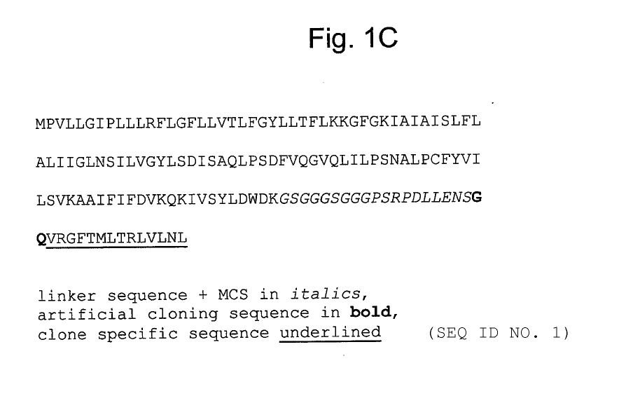

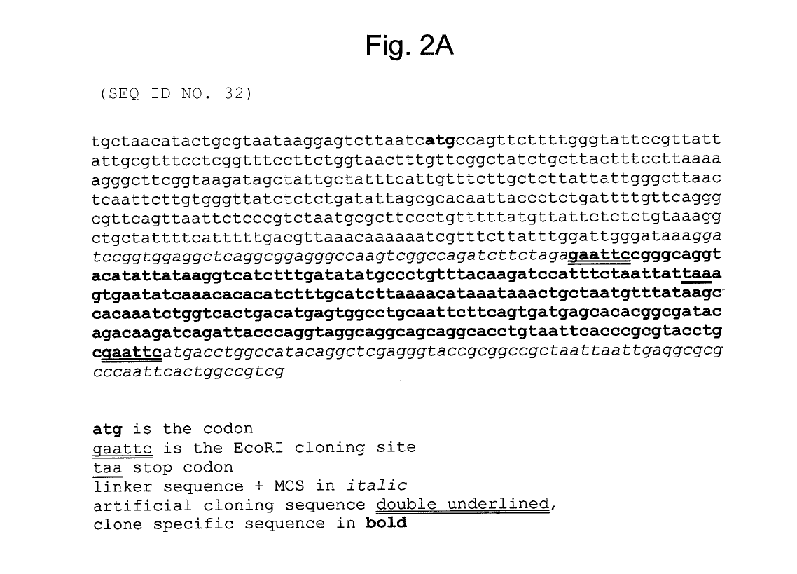

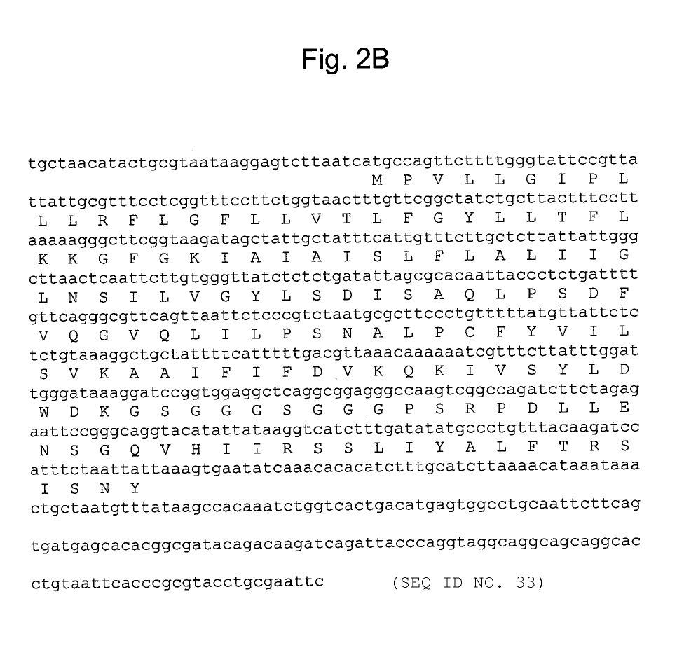

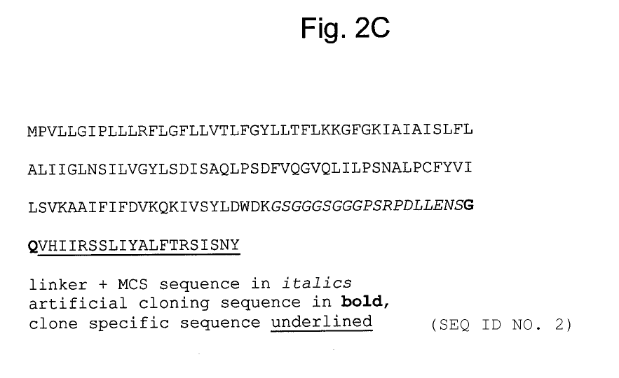

Sequence analysis revealed that these four clones represented two different antigenic peptides. The inventors then further focused on the two antigens, E1 (FIGS. 1a-c) and E12 (FIGS. 2a-c), that were only reactive with ruptured sera. Clones E1 and E12 were tested on a large panel of individual sera (n=38 for ruptured sera, n=23 for stable sera and n=10 for normal sera).

Clone E1 showed significant reactivity (ratio mean OD₄₅₀ sample/(mean OD₄₅₀ empty phage+3*SD)>1) to 22 out of 38 ruptured sera (58%) whereas no reactivity towards the stable and age and sex matched control sera tested was observed.

In addition, clone E12 showed enhanced reactivity in 15 out of 38 patient sera (40%), while none of the stable and control sera tested positive (see FIGS. 11A and B).

Combination of the serum reactivity against clones E1 and E12 even further increased the sensitivity to 74%, while the specificity remained at 100% (FIG. 11C).

To determine whether the observed positive reaction of ruptured sera was indeed due to the ruptured plaque derived sequences, five ruptured sera were pre-incubated with the synthetic peptides Ac-E1-Lys-NH₂ (Ac-VRGFTMLTRLVLNLK-NH₂)SEQ ID NO: 1 and Ac-E12-Lys-NH₂ (Ac-VHIIRSSLIYALFTRSISNYK-NH₂)SEQ ID NO: 2, representing the cDNA inserts of clones E1 and E12, and with a non-specific peptide.

As shown in FIG. 12A, pre-incubation with peptides Ac-E1-Lys-NH₂ and Ac-E12-Lys-NH₂, clearly inhibited the formation of specific IgG antibody/phage E1 and IgG antibody/phage E12 complexes. In contrast, the serum reactivity against clones E1 and E12 was not inhibited by addition of the random peptide.

Since testing the sera against the combination of clones E1 and E12 raised the sensitivity of the assay to 74%, it was tested whether inclusion of additional phage-displayed peptides would further increase the sensitivity. To this end, a panel of 192 randomly chosen recombinant phages from selection round 4 was tested with six ruptured, six stable and six control sera. Of the 192 clones tested, 118 phage-displayed peptides showed a positive reaction with at least one ruptured serum, while 106 clones interacted solely with one or more ruptured sera.

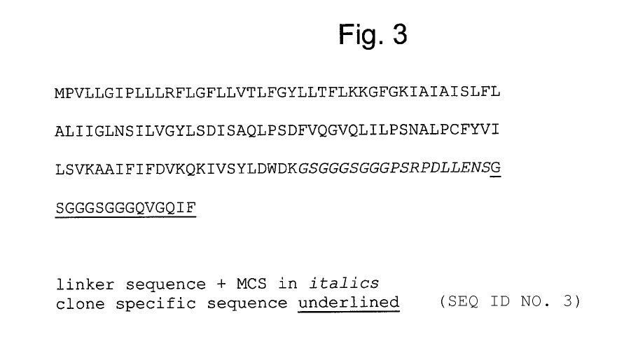

The panel of 106 clones comprised seven different recombinant phages that led to 100% sensitivity and 100% specificity for the identification of ruptured sera. Table 2 lists the additional peptides that specifically react with sera from patients suffering from ruptured plaques. The sequences are also shown in FIGS. 3-7.

TABLE 2

| clone | | amino acid sequence |
|---|---|---|
| mp1 | (SEQ ID NO: 3) | GSGGGSGGGQVGQIF |
| mp23 | (SEQ ID NO: 4) | RYKDFVNVIQGPQHPCVCRVPSKLSCSS |
| | | RCQPVNFPPYPRI |
| mp36 | (SEQ ID NO: 5) | REFITRYV |
| mp64 | (SEQ ID NO: 6) | EFRAGTTALVLDYWNNIQPRTEKLNRLT |
| | | IVCSLDGLTASHLPEPFQNEQRSPHHYV |
| | | FFFDKFRREEMCVPRGGEPDHTGSRVPP |
| | | KKKN |
| mp179 | (SEQ ID NO: 7) | NQKLNTIGNW |

The invention thus relates to peptides that interact with ruptured atherosclerotic plaque-associated antibodies in an individual, the peptides comprising an immunoreactive part of one of the following amino acid sequences:

SEQ ID NO: 1:
VRGFTMLTRLVLNL
SEQ ID NO: 2:
VHIIRSSLIYALFTRSISNY
SEQ ID NO: 3:
GSGGGSGGGQVGQIF
SEQ ID NO: 4:
RYKDFVNVIQGPQHPCVCRVPSKLSCSSRCQPVNFPPYPRICYSPNHFPV
SFQ
SEQ ID NO: 5:
REFITRYV
SEQ ID NO: 6:
EFRAGTTALVLDYWNNIQPRTEKLNRLTIVCSLDGLTASHLPEPFQNEQR
SPHHYVFFFDKFRREEMCVPRGGEPDHTGSRVPPKKKN
SEQ ID NO: 7:
NQKLNTIGNW

In a further embodiment the invention relates to antigens, comprising an immunoreactive part of one of the following amino acid sequences:

SEQ ID NO: 8:
GQVRGFTMLTRLVLNL
SEQ ID NO: 9:
GQVHIIRSSLIYALFTRSISNY
SEQ ID NO: 10:
SSREFITRYV
SEQ ID NO: 11:
SSREFRAGTTALVLDYWNNIQPRTEKLNRLTIVCSLDGLTASHLPEPFQN
EQRSPHHYVFFFDKFRREEMCVPRGGEPDHTGSRVPPKKKN

In another embodiment the invention relates to antigens, comprising an immunoreactive part of one of the following amino acid sequences:

SEQ ID NO: 12:
GSGGGSGGGPSRPDLLENSGQVRGFTMLTRLVLNL
SEQ ID NO: 13:
GSGGGSGGGPSRPDLLENSGQVHIIRSSLIYALFTRSISNY
SEQ ID NO: 14:
GSGGGSGGGPSRPDLLENSGSGGGSGGGQVGQIF
SEQ ID NO: 15:
GSGGGSGGGPSRPDLLENSRYKDFVNVIQGPQHPCVCRVPSKLSCSSRCQ
PVNFPPYPRICYSPNHFPVSFQ
SEQ ID NO: 16:
GSGGGSGGWAKSARSSREFITRYV
SEQ ID NO: 17:
GSGGGSGGWAKSARSSREFRAGTTALVLDYWNNIQPRTEKLNRLTIVCSL
DGLTASHLPEPFQNEQRSPHHYVEFFDKFRREEMCVPRGGEPDHTGSRVP
PKKKN
SEQ ID NO: 18:
GSGGGSGGGPSRPDLLENSNQKLNTIGNW

In a still further embodiment the invention relates to antigens, comprising an immunoreactive part of one of the following amino acid sequences:

SEQ ID NO: 19:
MPVLLGIPLLLRFLGFLLVTLFGYLLTFLKKGFGKIAIAISLFLALTIGL
NSILVGYLSDISAQLPSDFVQGVQLILPSNALPCFYVILSVKAAIFTFDV
KQKIVSYLDWDKGSGGGSGGGPSRPDLLENSGQVRGFTMLTRLVLNL
SEQ ID NO: 20:
MPVLLGTPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSGQVHIIRSSLIYALFTRSI
SNY
SEQ ID NO: 21:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFTFDV
KQKIVSYLDWDKGSGGGSGGGPSRPDLLENSGSGGGSGGGQVGQIF
SEQ ID NO: 22:
MPVLLGTPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSRYKDFVNVIQGPQHPCVCR
VPSKLSCSSRCQPVNFPPYPRICYSPNHFPVSFQ
SEQ ID NO: 23:
MPVLLGIPLLLRFLGFLLVTLFGYLLTFLKKGFGKIAIAISLELALIIGL
NSILVGYLSDISAQLPSDFVQGVQLILPSNALPCFYVILSVKAAIFTFDV
KQKIVSYLDWDKGSGGGSGGWAKSARSSREFITRYV
SEQ ID NO: 24:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGWAKSARSSREFRAGTTALVLDYWNNIQPRT
EKLNRLTIVCSLDGLTASHLPEPFQNEQRSPHHYVFFFDKFRREEMCVPR
GGEPDHTGSRVPPKKKN
SEQ ID NO: 25:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSNQKLNTIGNW

The part MPVLLGIPLLLRFLGFLLVTLFGYLLTFLKKGFGKIAIAISLFLALIIGLN SILVGYLSDISAQLPSDFVQGVQLILPSNALPCFYVILSVKAAIFIFDVKQKIVSYLDWDKGSG GGSGGGPSRPDLLENS in SEQ ID NOS: 19, 20, 22, 23 and 25 is derived from the pVI part and linker of pSP6B. The linker sequence is shown in bold. Amino acids shown in italics are derived from an artificial cloning sequence. The part MPVLLGIPLLLRFLGFLLVTLFGYLLTFLKKGFGKIAIAISLFLALIIGLNSILVG

YLSDISAQLPSDFVQGVQLILPSNALPCFYVILSVKAAIFIFDVKQKIVSYLDWDKG SGGGSGGWAKSAR in SEQ ID NOS 23-24 is derived from the pVI part and linker of vector pSP6C. The linker sequence is shown in bold. Amino acids shown in italics are derived from an artificial cloning sequence.

“Immunoreactive part” is defined herein as a part of the amino acid sequence according to SEQ ID NOS: 1-25 that shows enhanced reactivity to ruptured serum as compared to its reactivity with stable and normal serum. Such immunoreactive parts can be identified without undue burden with well-known techniques such as the PEPSCAN™ method.

“Enhanced reactivity” as used herein is in general any statistically significant difference between the reaction of a peptide according to the invention with ruptured sera and the reaction of the same peptide with stable and/or normal sera. More in particular “enhanced reactivity” means that the ratio (mean OD₄₅₀ sample/(mean OD₄₅₀ empty phage+X*SD) is >1. X is in particular more than one, more in particular at least 2, more in particular 3 or more.

“Ruptured serum” is serum from an individual or pooled serum of multiple individuals that have ruptured plaques. “Stable serum” is serum from an individual or pooled serum of multiple individuals that have stable plaques but no evidence of the presence of ruptured plaques. “Normal serum” is serum from an individual or pooled serum of multiple individuals that do not have clinical relevant cardiovascular disease.

Peptides of the invention comprise an amino acid sequence that is structurally identical to or functionally similar to an immunoreactive part of the amino acid sequences according to SEQ ID NOS: 1-25 or conservative variants thereof. The peptides may comprise further amino acids in addition to the amino acids that constitute the immunoreactive part and that may or may not contribute to the immunoreactivity of the complete peptide or may be present to facilitate binding of the peptides to a solid support, enable labelling or for other purposes. The amino acids that constitute the immunoreactive part are not necessarily consecutive amino acids.

Conservative variations of the peptides are also part of the present invention. The term “conservative variation” as used herein denotes the replacement of an amino acid residue by another, biologically similar residue. Examples of conservative variations include the substitution of one hydrophobic residue, such as isoleucine, valine, leucine or methionine, for another, or the substitution of one polar residue for another, such as the substitution of arginine for lysine, glutamic acid for aspartic acid, or glutamine for asparagine, and the like.

The term “conservative variation” also includes the use of a substituted amino acid in place of an unsubstituted parent amino acid and/or of a non-natural amino acid in place of a natural amino acid provided that antibodies to the peptide comprising the substituted and/or natural amino acid(s) also immunoreact with the peptide comprising the unsubstituted and/or non-natural amino acid(s). By using a routine screening method, such as by testing a conservative variant antigen with sera from a patient that is known to suffer from ruptured plaques, one of skill in the art can readily determine if the variant peptide has the requisite biological activity of the peptide of the invention without having to resort to undue experimentation.

According to a further aspect thereof the invention relates to a diagnostic reagent for the detection of ruptured atherosclerotic lesions in an individual, wherein said reagent comprises at least one peptide, said peptide comprising an immunoreactive part of the amino acid sequence as shown in any one of the SEQ ID NOS:1-25.

In preferred embodiments of the invention, the diagnostic reagent comprises combinations of peptides to increase the sensitivity as compared to the use of only one peptide. Suitable combinations are peptides comprising an immunoreactive part of the amino acid sequence as shown in any one of the SEQ ID NOS:1, 8, 12 and 19 with peptides comprising an immunoreactive part of the amino acid sequence as shown in any one of the SEQ ID NOS:2, 9, 13 and 20. In a further embodiment one or more peptides are added to this mixture that comprise an immunoreactive part of the amino acid sequence as shown in any one of the SEQ ID NOS:3-7, 10-11, 14-18 and 21-25.

Alternatively, a peptide comprising an immunoreactive part of the amino acid sequence as shown in any one of the SEQ ID NOS: 1, 8, 12 and 19 is combined with one or more peptides that comprise an immunoreactive part of the amino acid sequence as shown in any one of the SEQ ID NOS: 3-7, 10-11, 14-18 and 21-25.

In another alternative embodiment, a peptide comprising an immunoreactive part of the amino acid sequence as shown in any one of the SEQ ID NOS: 2, 9, 13 and 20 is combined with one or more peptides that comprise an immunoreactive part of the amino acid sequence as shown in any one of the SEQ ID NOS: 3-7, 10-11, 14-18 and 21-25.

Each combination of peptides can furthermore comprise more than one peptide that comprises an immunoreactive part of the amino acid sequence as shown in any one of the SEQ ID NOS: 1, 8, 12 and 19, and/or more than one peptide that comprises an immunoreactive part of the amino acid sequence as shown in any one of the SEQ ID NOS: 2, 9, 13 and 20 and/or more than one peptide that comprises an immunoreactive part of the amino acid sequence as shown in any one of the SEQ ID NOS: 3-7, 10-11, 14-18 and 21-25.

The above combinations of peptides can be considered a panel of peptides that can be used for determining a fingerprint of antibodies present in an individual suffering from ruptured atherosclerotic lesions.

According to the invention, a sample is positive when there is a reaction between antibodies in the sample to be tested with at least one peptide of the invention.

The peptides of the invention can be isolated peptides that comprise at least the immunoreactive part of any one of the SEQ ID NOS: 1-25. However, these peptides or immunoreactive parts thereof may still be present in the phage. Thus the antigen used for diagnosing the presence in an individual of ruptured atherosclerotic lesions may be isolated peptide but also phage-displayed peptide or phage lysate containing peptide of the invention. The phage may be any filamentous phage and is in particular M13 or a variant thereof.

According to a further aspect thereof the invention relates to a method for diagnosing a sample from an individual for the presence therein of antibodies that are indicative of the presence of ruptured atherosclerotic plaques in the individual, wherein the sample is contacted with the peptide or the diagnostic reagent and immune complexes formed between said peptide or reagent and the antibodies are detected.

In a further embodiment the invention relates to a test kit for the diagnosis of a sample from an individual for the presence therein of antibodies that are indicative of the presence of ruptured atherosclerotic plaques in the individual, wherein the test kit comprises one or more antigens or the diagnostic reagent of the invention and optionally means for visualizing formation of immunecomplexes.

The sample from the individual to be tested can be a body fluid, such as blood, serum, plasma etc. or a tissue sample, in particular atherosclerotic plaque tissue. Antibody present in the sample will bind to the antigen. After removal of the unbound antigen the antigen-antibody complex can be determined and quantitated by means of various known techniques.

In a first embodiment, a predetermined quantity of antigen (which is the peptide or diagnostic reagent) is adsorbed on a solid phase protein-binding surface. The test sample to be assayed for antibodies is then contacted with the surface having the antigen bound thereto. Antibodies in the test sample bind to the immobilized antigen thus forming an antigen-antibody complex. The complex is subsequently detected by any detecting agent such as an antibody-enzyme conjugate that binds to the immobilized antibody in the antigen-antibody complex. A substrate that changes colour when acted on by the enzyme is used to quantify the amount of bound antibody-enzyme conjugate, which is indicative of the antibody concentration in the test sample. Alternatively, the detecting agent that binds to the antibody in the antigen-antibody complex is a radioactively labelled agent, in particular an antibody. The amount of bound detecting agent is then quantitated by means of the amount of bound radioactivity.

In another embodiment immunecomplexes between the antigen and the antibodies to be detected are formed and then separated from non-bound antigen and antibodies, for example by capturing the complexes on a solid support, and quantitated.

A solid support can be any solid support that is used in diagnostic assays of the type described above and can be for example, the inner wall of a microtest well or a cuvette, a tube or capillary, a membrane, filter, test strip or the surface of a particle such as, for example, a latex particle, an erythrocyte, a dye sol, a metal sol or metal compound as sol particle, a carrier protein such as bovine serum albumin (BSA) or keyhole limpet hemocyanin (KLH).

In a particular embodiment, a diagnostic reagent of the invention may thus comprise one or more peptides in any form, i.e. as isolated peptides, as phage-displayed peptides, as peptides in phage lysates etc., and a suitable support. A test kit of the invention comprises in a particular embodiment such diagnostic reagent and a labelling substance for visualizing the immunocomplexes between the peptides of the invention and the ruptured-plaque associated antibodies in the sera to be tested.

Labelling substances which can be used are, inter alia, a radioactive isotope, a fluorescent compound, a chemiluminescent compound, an enzyme, a dye sol, metal sol or metal compound as sol particle, etc. Examples of enzymatic labelling systems comprise the peroxidase/anti-peroxidase (PAP) system, the alkaline phosphatase/anti-alkaline phosphatase (APAAP) system, the avidin-biotin and biotin-streptavidin system.

Depending on the nature and further characteristics of the reagents, the immunochemical reaction that takes place is a so-called sandwich reaction, an agglutination reaction, a competition reaction, an inhibition reaction or other assay formats. These assay formats are well-known in the art.

The techniques for detecting the formation of immunocomplexes in a sample are well known and all fall within the scope of this invention which is based on the use of the newly identified peptides that bind specifically to ruptured plaque-associated antibodies. “Ruptured plaque-associated antibodies” as used herein are antibodies that are present in serum from an individual that suffers from ruptured atherosclerotic plaques and can be detected therein by binding to the peptides of the invention. Normal serum or serum from an individual having stable plaques does not have a significant detectable amount of antibodies that specifically bind to these peptides.

The present invention will be further illustrated in the examples that follow and that are not intended to limit the invention in any way. In the description and examples reference is made to the following figures.

FIG. 1A (SEQ ID NO. 30) shows the nucleotide sequence of clone E1.

FIG. 1B (SEQ ID NO. 31) shows the amino acid sequence of clone E1.

FIG. 1C (SEQ ID NO. 1) shows the full length fusion protein of clone E1.

FIG. 2A (SEQ ID NO. 32) shows the nucleotide sequence of clone E12.

FIG. 2B (SEQ ID NO. 33) shows the amino acid sequence of clone E12.

FIG. 2C (SEQ ID NO. 2) shows the full length fusion protein of clone E12.

FIG. 3 (SEQ ID NO. 3) shows the full length fusion protein of clone mp1.

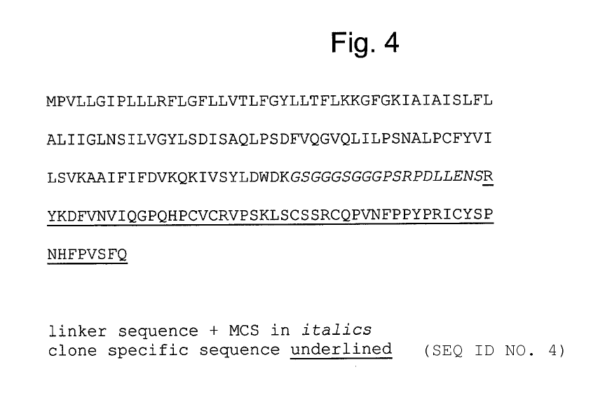

FIG. 4 (SEQ ID NO. 4) shows the full length fusion protein of clone mp23.

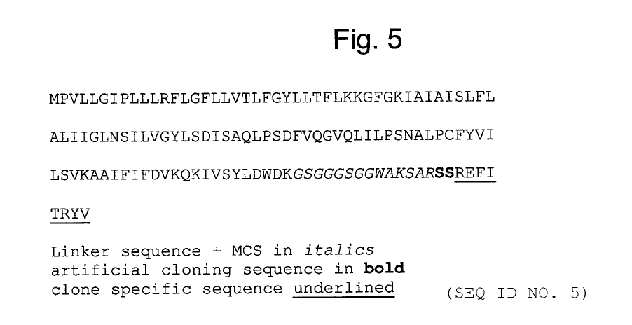

FIG. 5 (SEQ ID NO. 5) shows the full length fusion protein of clone mp36.

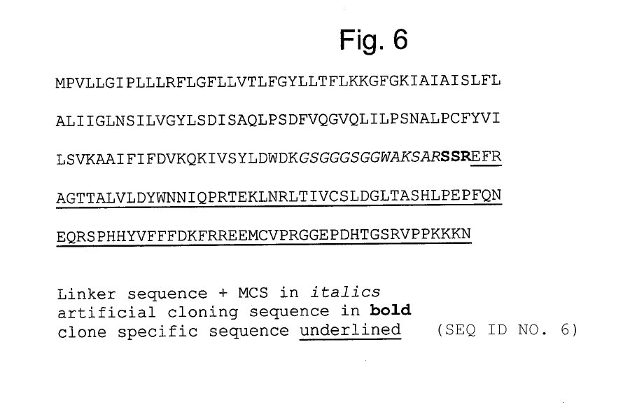

FIG. 6 (SEQ ID NO. 6) shows the full length fusion protein of clone mp64.

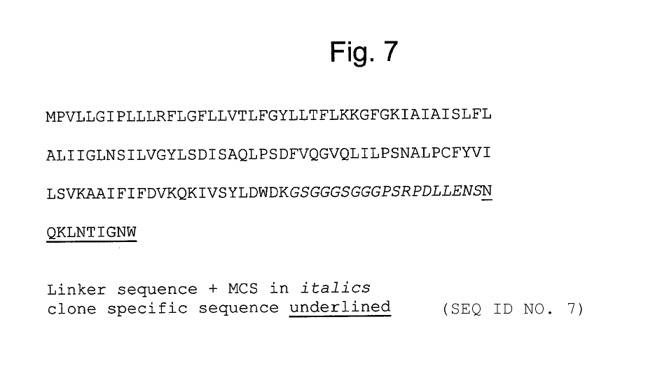

FIG. 7 (SEQ ID NO. 7) shows the full length fusion protein of clone mp179.

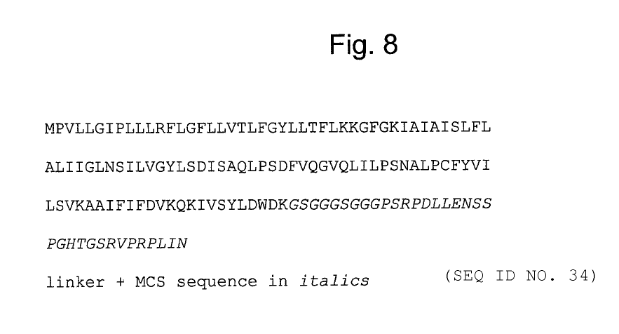

FIG. 8 (SEQ ID NO. 34) shows the nucleotide sequence and amino acid sequence of the empty phage display vector pSP6B with the pVI sequence, the linker and the multiple cloning sequence.

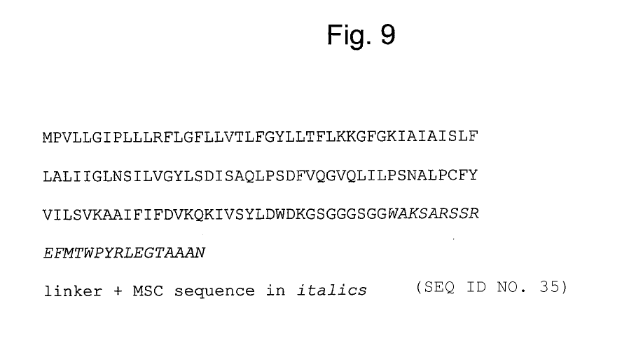

FIG. 9 (SEQ ID NO. 35) shows the nucleotide sequence and amino acid sequence of the empty phage display vector pSP6C with the pVI sequence, the linker and the multiple cloning sequence.

FIG. 10 shows the reactivity of different antigens to pooled ruptured serum. Reactivity is represented as the ratio of mean OD₄₅₀ sample/(mean OD₄₅₀ empty phage+3*SD). #=clones with identical inserts.

FIG. 11(A) shows the serum reactivity to clone E1. FIG. 11(B) shows the serum reactivity to clone E12. FIG. 11(C) shows the sensitivity and specificity of clones E1 and E12.

FIGS. 12(A)-(B) shows the ability of synthetic peptides Ac-E1-Lys-NH₂ and Ac-E12-Lys-NH₂ to deplete human ruptured sera from antibodies directed against phage E1(A) and E12(B), respectively. Reactivity is represented as the ratio of mean OD₄₅₀ sample/(mean OD₄₅₀ empty phage+3*SD).

### EXAMPLES

### Example 1

ELISA of Antigens of the Invention on Pooled Plasma

Two clones of a phage displayed ruptured plaque enriched cDNA expression library were used in an ELISA to test the reactivity of these clones with pooled patient sera. To select the clones an expression library of cDNAs predominantly expressed in ruptured atherosclerotic lesions, a SSH library of over 3000 ruptured plaque enriched cDNAs (described in Faber et al. Circ Res. 89:547-554 (2001)) was used. This library was re-cloned into the phage display vectors pSP6A, B and C (Hufton et al. J Immunol Methods. 231: 39-51 (1999)). Using these vectors, the SSH cDNA fragments are expressed as a fusion protein with the minor coat protein pVI of filamentous phage M13 in all reading frames.

After various rounds of selection with pooled sera from patients known to suffer from ruptured atherosclerotic plaques (=ruptured sera) the two clones E1 and E12 as shown in FIGS. 1 and 2 were selected because they showed enhanced reactivity (ratio mean OD₄₅₀ sample/mean OD₄₅₀ empty phage+3×SD) to pooled ruptured serum (FIG. 10; background=empty phage).

Selection was performed in a 96-well flat-bottom micro-titer plate (Falcon, Franklin Lakes, N.J.) coated o/n at 4° C. with 200 μl rabbit anti-human IgG (10 μg/ml in coating buffer)/well. Subsequently, the plate was washed twice with PBST (0.1% (v/v) Tween 20 in PBS) and twice with PBS, blocked with 2% MPBS (2% Marvel (=skimmed milk) in PBS) for 2 hrs at RT, and washed again 3 times with PBST and 3 times with PBS. In a 96 wells round bottom plate (Costar, Corning, N.Y.), 50 μl pre-adsorbed pooled serum (by repeated passage through a column of Sepharose 6 MB (Pharmacia, Uppsala, Sweden) coupled to E. coli TG1 and bacteriophage infected E. coli TG1 lysates to remove antibodies reactive to antigens related to the phage-host infection; diluted 1:100 in 2% MPBS) was added to 100 μl crude phage supernatant diluted 1:1 in 4% MPBS.

The mixture was incubated at 37° C. for 1 hr followed by a 30 min incubation at RT. Next, the pre-incubated serum-phage mixture was transferred to the blocked and washed rabbit anti-human IgG coated micro-titer plate and incubated at 37° C. for 1 hr followed by 30 min incubation at RT.

After washing, 150 μl (1:5000 in 2% MPBS) horseradish peroxidase (HRP) conjugated anti-phage (anti-M13) monoclonal antibody (Amersham, Uppsala, Sweden) was added and incubated 1 hr at RT. After washing, 100 μl 3,3′,5,5′-tetramethyl-benzidine dihydrochloride chromogen (TMB) solution (10 mg/ml) was added to each well. The reaction was stopped by the addition of 50 μl 2N H₂SO₄/well.

Plates were read at 450 nm in a Novapath micro-titer plate reader (Biorad, Hercules, Calif.).

ELISA using individual patient sera was essentially as described above. However, adsorption of sera against bacterial and phage-related proteins was not performed, as no difference in background signals between adsorbed and non-adsorbed sera was observed. Each determination was done in triplicate. Both the intra- and interassay variability were <5%.

Four clones (A1, A7, E1 and E12) showed enhanced reactivity (ratio mean OD₄₅₀ sample/mean OD₄₅₀ empty phage+3*SD) to pooled ruptured serum as compared to their reactivity with stable and normal serum (see FIG. 10). These antigens thus showed a ruptured plaque specific antibody signature. Furthermore, both dilution of sera and decreasing the amount of recombinant phages resulted in diminished ELISA reactivity, clearly indicating the specificity of the observed interaction (data not shown).

### Example 2

Detailed Serological Analysis of Antigens E1 and E12

Clones E1 and E12 were tested on a large panel of individual sera (n=38 for ruptured sera, n=23 for stable sera and n=10 for normal sera). Table 1 shows detailed patient characteristics.

Serum samples were obtained from patients undergoing peripheral vascular surgery (Department of General Surgery, Academic Hospital Maastricht) and stored at −20EC. Control sera (n=10) were derived from age and sex matched blood donors (n=10) from the Dutch blood bank (Sanquin).

TABLE 1

| | Ruptured | Stable |
|---|---|---|
| | (n = 38) | (n = 23) |
| Age (mean +/− SEM) | 64.5 ± 1.7 | 64.6 ± 2.0 |
| Male (%) | 76% | 61% |
| Hypertensive (%) | 41% | 50% |
| Smoker (%) | 52% | 37% |
| Diabetic (%) | 47% | 52% |
| Statin use (%) | 50% | 46% |
| CRP (mean +/− SEM) | 9.2 ± 2.0 | 6.2 ± 2.0 |
| Cholesterol (mean +/− SEM) | 5.7 ± 0.2 | 6.1 ± 0.3 |
| LDL (mean +/− SEM) | 4.0 ± 0.2 | 4.0 ± 0.3 |
| HDL (mean +/− SEM) | 1.2 ± 0.1 | 1.2 ± 0.1 |
| Triglycerydes (mean +/− SEM) | 2.1 ± 0.2 | 2.0 ± 0.3 |

Clone E1 showed significant reactivity to 22 out of 38 ruptured sera (58%) whereas no reactivity towards the stable and age and sex matched control sera tested was observed. In addition, clone E12 showed enhanced reactivity in 15 out of 38 patient sera (40%), while again none of the stable and control sera tested positive (see FIGS. 11A and B).

Combination of the serum reactivity against clones E1 and E12 even further increased the sensitivity to 74%, while the specificity remains at 100% (FIG. 11C).

### Example 3

Serological Analysis of a Ruptured Plaque Specific Ag Panel

The combination of reactivity against clones E1 and E12 raised the sensitivity of the assay to 74%. Therefore, it was tested whether extension of the amount of clones included would further increase the sensitivity. To this end, 6 ruptured sera, 6 stable sera and 6 normal sera were tested for there reactivity to a panel of 192 randomly chosen recombinant phages from selection round 4. With this panel of 192 clones a 100% sensitivity and 100% specificity for the identification of ruptured sera was obtained.

Of the clones that reacted solely with one or more ruptured sera were identical to clone E1, while 36 clones were identical to clone E12. Another 15 clones interacted also solely with ruptured sera. These 15 clones had five different amino acid sequences as given in Table 2.

### Example 4

Inhibition of Reactivity of Ruptured Sera with Synthetic Peptides

In order to determine whether the observed reactivity of ruptured sera was due to the clone specific, ruptured plaque derived, cDNA insert sequences, 5 ruptured sera were pre-incubated with the synthetic peptides E1 (Ac-VRGFTMLTRLVLNLK-NH₂) SEQ ID NO: 1 and E12 (Ac-VHIIRSSLIYALFTRSISNYK-NH₂) SEQ ID NO:2 representing the clone specific part of the protein VI-ruptured plaque specific antigen fusion proteins expressed by phage clones E1 and E12 and with a non-specific peptide.

Synthetic peptides Ac-E1-Lys-NH₂ and Ac-E12-Lys-NH₂ were prepared by standard manual solid-phase peptide synthesis and subsequent purification by reversed-phase HPLC.

Fifteen μl serum of 5 individual patients were pre-incubated for 20 min at 37° C., 20 min at RT and 20 min at 4° C. in the presence of 50 ng of random peptide, synthetic peptide Ac-E1-Lys-NH₂ (Ac-VRGFTMLTRLVLNLK-NH₂) SEQ ID NO:1 or Ac-E12-Lys-NH₂ (Ac-VHIIRSSLIYALFTRSISNYK-NH₂) SEQ ID NO:2. Subsequently, peptide/IgG complexes were precipitated by spinning at 20,000 g and discarded. After two such depletion rounds, the reactivity to phage E1 and E12 was determined in ELISA as described above.

As is shown in FIG. 12, pre-incubation with the Ac-E1-Lys-NH₂ (“peptide E1”) and Ac-E12-Lys-NH₂ (“peptide E12”) peptides, clearly inhibited the formation of specific IgG antibody/phage E1 and IgG antibody/phage E12 complexes, while the serum reactivity against clones E1 and E12 was not inhibited by addition of the random peptide.

1. Peptides that are able to interact with ruptured atherosclerotic plaque-associated antibodies, which peptides comprise an immunoreactive part of one of the following amino acid sequences:
SEQ ID NO: 1:
VRGFTMLTRLVLNL
SEQ ID NO: 2:
VHIIRSSLIYALFTRSISNY
SEQ ID NO: 3:
GSGGGSGGGQVGQIF
SEQ ID NO: 4:
RYKDFVNVIQGPQHPCVCRVPSKLSCSSRCQPVNFPPYPRICYSPNHFPV
SFQ
SEQ ID NO: 5:
REFITRYV
SEQ ID NO: 6:
EFRAGTTALVLDYWNNIQPRTEKLNRLTIVCSLDGLTASHLPEPFQNEQR
SPHHYVFFFDKFRREEMCVPRGGEPDHTGSRVPPKKKN
SEQ ID NO: 7:
NQKLNTIGNW.

2. Peptides as claimed in claim 1, comprising an immunoreactive part of one of the following amino acid sequences:
SEQ ID NO: 8:
GQVRGFTMLTRLVLNL
SEQ ID NO: 9:
GQVHIIRSSLIYALFTRSISNY
SEQ ID NO: 10:
SSREFITRYV
SEQ ID NO: 11:
SSREFRAGTTALVLDYWNNIQPRTEKLNRLTIVCSLDGLTASHLPEPFQN
EQRSPHHYVFFFDKFRREEMCVPRGGEPDHTGSRVPPKKKN.

3. Peptides as claimed in claim 1 or 2, comprising an immunoreactive part of one of the following amino acid sequences:
SEQ ID NO: 12:
GSGGGSGGGPSRPDLLENSGQVRGFTMLTRLVLNL
SEQ ID NO: 13:
GSGGGSGGGPSRPDLLENSGQVHIIRSSLIYALFTRSISNY
SEQ ID NO: 14:
GSGGGSGGGPSRPDLLENSGSGGGSGGGQVGQIF
SEQ ID NO: 15:
GSGGGSGGGPSRPDLLENSRYKDFVNVIQGPQHPCVCRVPSKLSCSSRCQ
PVNFPPYPRICYSPNHFPVSFQ
SEQ ID NO: 16:
GSGGGSGGWAKSARSSREFITRYV
SEQ ID NO: 17:
GSGGGSGGWAKSARSSREFRAGTTALVLDYWNNIQPRTEKLNRLTIVCSL
DGLTASHLPEPFQNEQRSPHHYVFFFDKFRREEMCVPRGGEPDHTGSRVP
PKKKN
SEQ ID NO: 18:
GSGGGSGGGPSRPDLLENSNQKLNTIGNW.

4. Peptides as claimed in any one of the claims 1-3, comprising an immunoreactive part of one of the following amino acid sequences:
SEQ ID NO: 19:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSGQVRGFTMLTRLVLNL
SEQ ID NO: 20:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSGQVHIIRSSLIYALFTRSI
SNY
SEQ ID NO: 21:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSGSGGGSSSSQVSQIF
SEQ ID NO: 22:
MPVLLSIPLLLRFLSFLLVTLFSYLLTFLKKSFSKIAIAISLFLALIISL
NSILVSYLSDISAQLPSDFVQGVQLILPSNALPCFYVILSVKAAIFIFDV
KQKIVSYLDWDKGSGGGSGGGPSRPDLLENSRYKDFVNVIQGPQHPCVCR
VPSKLSCSSRCQPVNFPPYPRICYSPNHFPVSFQ
SEQ ID NO: 23:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGWAKSARSSREFITRYV
SEQ ID NO: 24:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGWAKSARSSREFRAGITALVLDYWNNIQPRT
EKLNRLTIVCSLDGLTASHLPEPFQNEQRSPHHYVFFFDKFRREEMCVPR
GGEPDHTGSRVPPKKKN
SEQ ID NO: 25:
MPVLLGIPLLLRFLGFLLVTLFGYLLTFLKKGFGKIAIAISLFLALIIGL
NSILVGYLSDISAQLPSDFVQGVQLILPSNALPCFYVILSVKAAIFIFDV
KQKIVSYLDWDKGSGGGSGGGPSRPDLLENSNQKLNTIGNW.

5. Peptides as claimed in any one of the claims 1-4 for use in diagnosis.

6. Peptides as claimed in claim 5, wherein the use is for the diagnosis of the presence of ruptured atherosclerotic plaques in an individual.

7. Peptides as claimed in claim 6, wherein the diagnosis comprises detection in a sample of body fluid or tissue of an individual of antibodies that are indicative for the presence of ruptured atherosclerotic plaques in the said individual by binding to one or more of the peptides.

8. Peptides as claimed in claim 7, wherein the sample of body fluid is blood, serum, plasma.

9. Peptides as claimed in claim 7, wherein the sample of tissue is atherosclerotic plaque tissue.

10. Use of one or more of the peptides as claimed in any one of the claims 1-4 for the preparation of a diagnostic reagent.

11. Use as claimed in claim 10, wherein the diagnostic reagent is for use in the diagnosis of the presence of ruptured atherosclerotic plaques in an individual.

12. Use as claimed in claim 11, wherein the diagnosis comprises detection in a sample of body fluid or tissue of an individual of antibodies that are indicative for the presence of ruptured atherosclerotic plaques in the said individual by binding to the diagnostic reagent.

13. Diagnostic reagent for the detection of ruptured atherosclerotic lesions in an individual, characterized in that said reagent comprises at least one peptide, comprising an immunoreactive part of the amino acid sequence as shown in any one of the SEQ ID NOS: 1-25.

14. Diagnostic reagent as claimed in claim 13, wherein the one or more peptides are bound to a solid phase.

15. Method for the diagnosis of a sample from an individual for the presence therein of antibodies that are indicative of the presence of ruptured atherosclerotic plaques in the individual, characterized in that said sample is contacted with a diagnostic reagent according to claim 13 or 14 and immunecomplexes formed between said reagent and the antibodies are detected.

16. Test kit for the diagnosis of a sample from an individual for the presence therein of antibodies that are indicative of the presence of ruptured atherosclerotic plaques in the individual, characterized in that said test kit comprises a diagnostic reagent according to claim 13 or 14 and optionally means for visualizing the formation of immunecomplexes.


SEQUENCE LISTING (35 sequences)

SEQ ID NO: 1 - protein, 14 aa, Artificial Sequence
  Synthetic peptide
VRGFTMLTRLVLNL

SEQ ID NO: 2 - protein, 20 aa, Artificial Sequence
  Synthetic peptide
VHIIRSSLIYALFTRSISNY

SEQ ID NO: 3 - protein, 15 aa, Artificial Sequence
  recombinant phage clone amino acid
GSGGGSGGGQVGQIF

SEQ ID NO: 4 - protein, 53 aa, Artificial Sequence
  recombinant phage clone amino acid
RYKDFVNVIQGPQHPCVCRVPSKLSCSSRCQPVNFPPYPRICYSPNHFPVSFQ

SEQ ID NO: 5 - protein, 8 aa, Artificial Sequence
  recombinant phage clone amino acid
REFITRYV

SEQ ID NO: 6 - protein, 88 aa, Artificial Sequence
  recombinant phage clone amino acid
EFRAGTTALVLDYWNNIQPRTEKLNRLTIVCSLDGLTASHLPEPFQNEQRSPHHYVFFFDKFRREEMCVPRGGEPDHTGSRVPPKKKN

SEQ ID NO: 7 - protein, 10 aa, Artificial Sequence
  recombinant phage clone amino acid
NQKLNTIGNW

SEQ ID NO: 8 - protein, 16 aa, Artificial Sequence
  Synthetic linker
GQVRGFTMLTRLVLNL

SEQ ID NO: 9 - protein, 22 aa, Artificial Sequence
  Synthetic linker
GQVHIIRSSLIYALFTRSISNY

SEQ ID NO: 10 - protein, 10 aa, Artificial Sequence
  Synthetic linker
SSREFITRYV

SEQ ID NO: 11 - protein, 91 aa, Artificial Sequence
  Synthetic linker
SSREFRAGTTALVLDYWNNIQPRTEKLNRLTIVCSLDGLTASHLPEPFQNEQRSPHHYVFFFDKFRREEMCVPRGGEPDHTGSRVPPKKKN

SEQ ID NO: 12 - protein, 35 aa, Artificial Sequence
  Synthetic linker
GSGGGSGGGPSRPDLLENSGQVRGFTMLTRLVLNL

SEQ ID NO: 13 - protein, 41 aa, Artificial Sequence
  Synthetic linker
GSGGGSGGGPSRPDLLENSGQVHIIRSSLIYALFTRSISNY

SEQ ID NO: 14 - protein, 34 aa, Artificial Sequence
  Synthetic linker
GSGGGSGGGPSRPDLLENSGSGGGSGGGQVGQIF

SEQ ID NO: 15 - protein, 72 aa, Artificial Sequence
  Synthetic linker
GSGGGSGGGPSRPDLLENSRYKDFVNVIQGPQHPCVCRVPSKLSCSSRCQPVNFPPYPRICYSPNHFPVSFQ

SEQ ID NO: 16 - protein, 24 aa, Artificial Sequence
  Synthetic linker
GSGGGSGGWAKSARSSREFITRYV

SEQ ID NO: 17 - protein, 105 aa, Artificial Sequence
  Synthetic linker
GSGGGSGGWAKSARSSREFRAGTTALVLDYWNNIQPRTEKLNRLTIVCSLDGLTASHLPEPFQNEQRSPHHYVFFFDKFRREEMCVPRGGEPDHTGSRVPPKKKN

SEQ ID NO: 18 - protein, 29 aa, Artificial Sequence
  Synthetic linker
GSGGGSGGGPSRPDLLENSNQKLNTIGNW

SEQ ID NO: 19 - protein, 147 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENSGQVRGFTMLTRLVLNL

SEQ ID NO: 20 - protein, 153 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENSGQVHIIRSSLIYALFTRSISNY

SEQ ID NO: 21 - protein, 146 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENSGSGGGSGGGQVGQIF

SEQ ID NO: 22 - protein, 184 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENSRYKDFVNVIQGPQHPCVCRVPSKLSCSSRCQPVNFPPYPRICYSPNHFPVSFQ

SEQ ID NO: 23 - protein, 136 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGWAKSARSSREFITRYV

SEQ ID NO: 24 - protein, 217 aa, Artificial Sequence [sequence omitted]

SEQ ID NO: 25 - protein, 141 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENSNQKLNTIGNW

SEQ ID NO: 26 - protein, 131 aa, Artificial Sequence
  Synthetic peptide
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENS

SEQ ID NO: 27 - protein, 126 aa, Artificial Sequence
  Synthetic linker
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGWAKSAR

SEQ ID NO: 28 - protein, 15 aa, Artificial Sequence
  Synthetic
VRGFTMLTRLVLNLK

SEQ ID NO: 29 - protein, 21 aa, Artificial Sequence
  Synthetic
VHIIRSSLIYALFTRSISNYK

SEQ ID NO: 30 - DNA, 777 bp, Artificial Sequence
  clone E1; Fig 1A
tgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaaggatccggtggaggctcaggcggagggccaagtcggccagatcttctagagaattccgggcaggtacggggtttcnccatgttgaccaggttggtcttgaacttgtaagctcaagcgaactgcccatctctgcctcccaaagtgctgggattacagacatgggccnctgcncccggccttcaaataacttttgtatgtaacattacaatattaataatttttttctattccatcccattttaattagtgagaaaaaataattaaaaagcncaatttcttagcnttagttttgattatgaaataataggaatagtatagtattttgtttgaattcatgacctggccatacaggctcgagggtaccgcggccgctaattaattgaggcgcgcccaattcactggccgtcg

SEQ ID NO: 31 - DNA, 703 bp, Artificial Sequence
  clone E1; Fig 1B
tgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaaggatccggtggaggctcaggcggagggccaagtcggccagatcttctagagaattccgggcaggtacggggtttcnccatgttgaccaggttggtcttgaacttgtaagctcaagcgaactgcccatctctgcctcccaaagtgctgggattacagacatgggccnctgcncccggccttcaaataacttttgtatgtaacattacaatattaataatttttttctattccatcccattttaattagtgagaaaaaataattaaaaagcncaatttcttagcnttagttttgattatgaaataataggaatagtatagtattttgtttgaattc

SEQ ID NO: 32 - DNA, 763 bp, Artificial Sequence
  Clone E12; fig. 2A
tgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaaggatccggtggaggctcaggcggagggccaagtcggccagatcttctagagaattccgggcaggtacatattataaggtcatctttgatatatgccctgtttacaagatccatttctaattattaaagtgaatatcaaacacacatctttgcatcttaaaacataaataaactgctaatgtttataagccacaaatctggtcactgacatgagtggcctgcaattcttcagtgatgagcacacggcgatacagacaagatcagattacccaggtaggcaggcagcaggcacctgtaattcacccgcgtacctgcgaattcatgacctggccatacaggctcgagggtaccgcggccgctaattaattgaggcgcgcccaattcactggccgtcg

SEQ ID NO: 33 - DNA, 689 bp, Artificial Sequence
  Clone E12; fig 2B
tgctaacatactgcgtaataaggagtcttaatcatgccagttcttttgggtattccgttattattgcgtttcctcggtttccttctggtaactttgttcggctatctgcttactttccttaaaaagggcttcggtaagatagctattgctatttcattgtttcttgctcttattattgggcttaactcaattcttgtgggttatctctctgatattagcgcacaattaccctctgattttgttcagggcgttcagttaattctcccgtctaatgcgcttccctgtttttatgttattctctctgtaaaggctgctattttcatttttgacgttaaacaaaaaatcgtttcttatttggattgggataaaggatccggtggaggctcaggcggagggccaagtcggccagatcttctagagaattccgggcaggtacatattataaggtcatctttgatatatgccctgtttacaagatccatttctaattattaaagtgaatatcaaacacacatctttgcatcttaaaacataaataaactgctaatgtttataagccacaaatctggtcactgacatgagtggcctgcaattcttcagtgatgagcacacggcgatacagacaagatcagattacccaggtaggcaggcagcaggcacctgtaattcacccgcgtacctgcgaattc

SEQ ID NO: 34 - protein, 146 aa, Artificial Sequence
  display vector pSP6B; fig. 8
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGGPSRPDLLENSSPGHTGSRVPRPLIN

SEQ ID NO: 35 - protein, 145 aa, Artificial Sequence
  display vector psP6C; fig. 9
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDKGSGGGSGGWAKSARSSREFMTWPYRLEGTAAAN